(19) United States
(12) Patent Application Publication
(10) Pub. No.: US 2004/0032957 A1
(43) Pub. Date: Feb. 19, 2004

(54) Sensors and sensor assemblies for monitoring biological sounds and electric potentials

(76) Inventors: Hansen A. Mansy, Justice, IL (US); Richard H. Sandler, Evanston, IL (US)

Correspondence Address: Mark G Hanley, Grossman & Flight LLC, Suite 4220 20 North Wacker Drive, Chicago, IL, 60606, US

(21) Appl. No.: 10/218,298

(22) Filed: Aug. 14, 2002

(51) Int. Cl. A61B 7/04; A61B 5/02

(52) U.S. Cl. 381/67; 600/500

(57) ABSTRACT

A sensor for use with a biological entity includes a housing and an acoustic transducer disposed within the housing. The acoustic transducer is adapted to detect a biological sound impinging on a surface of the biological entity. The sensor may also include an electrode integral with the sensor. The electrode is adapted to detect an electric potential associated with the surface of the biological entity. A plurality of sensors can be held in a predetermined pattern on the surface of the biological entity using a flexible carrier that provides a plurality of sensor mounting locations.

### BACKGROUND

1. Field of the Invention

The invention relates generally to sensors for detecting properties of a biological entity and, more particularly, the invention relates to sensors and sensor assemblies for detecting biological sounds and electric potentials.

2. Description of Related Technology

Auscultation is a widely used diagnostic procedure that provides a high degree of diagnostic power, is readily available, is non-invasive and can be performed at a relatively low cost. Typically, auscultation is performed using a stethoscope that acquires and conveys sounds or vibrations from the surface of a patient's body to an examiner's ear. Historically, stethoscopes were primarily mechanical devices. However, more recent advances have resulted in electronic stethoscopes. Electronic stethoscopes are typically based on a transducer such as an electret microphone, an accelerometer, an optical sensor, or any other sensor that is capable of converting sounds or vibrations into electrical signals. Additionally, the detection capabilities of electronic stethoscopes are not inherently limited as are mechanical stethoscopes, which rely solely on human hearing. As is well known, the effectiveness of human hearing varies substantially as a function of frequency and amplitude of the sounds to be detected. For example, the sensitivity of human hearing is typically about 120 decibels lower at 20 Hertz (Hz) than at 1000 Hz. As a result, human hearing provides limited diagnostic capabilities because certain low frequency and/or low amplitude sounds that are useful for diagnostic purposes may be undetectable by humans.

Electronic stethoscopes in combination with recent advances in digital signal processing, telemedicine and other computer-related technologies have resulted in increased interest in the development of systems that can automatically acquire and analyze biological sounds or vibrations. Applications for electronic stethoscopes vary widely and include, for example, phonocardiology, phonopneumography and phongastroenterology.

Unfortunately, electronic stethoscopes are typically expensive devices that may require cleaning or sterilization after contact with a patient's skin. In other words, electronic stethoscopes are not generally disposable and do not typically provide a disposable portion that may be removed and replaced after each use. Furthermore, because patients in an emergency room or intensive care environment, and all patients receiving anesthesia, are routinely fitted with a plurality of electrocardiogram leads, the attachment of additional types of sensors (e.g., one or more acoustic sensors) to these patients is usually avoided. Although the attachment of acoustic sensors to patients may provide some useful diagnostic information to a physician or other operator, the use of acoustic sensors is often avoided in order to, at least in part, improve patient comfort and to eliminate the burden of having to attach additional sensors and wires to a patient's skin (i.e., in addition to the EKG sensors and wires that are usually attached).

### SUMMARY

In accordance with one aspect of the invention, a sensor for use with a biological entity may include a housing and a first acoustic transducer disposed within the housing and adapted to detect a biological sound impinging on a surface of the biological entity. In addition, the sensor may include an electrode integral with the sensor and adapted to detect an electric potential associated with the surface of the biological entity.

In accordance with another aspect of the invention, a sensor assembly for use with a biological entity may include a flexible carrier that is adapted to be attached to a surface of the biological entity. The sensor assembly may include a plurality of acoustic sensors fixed to the flexible carrier and defining a pattern within an area defined by a perimeter of the flexible carrier. Each of the acoustic sensors may be adapted to detect a sound at the surface of the biological entity.

In accordance with yet another aspect of the invention, a flexible carrier for holding a plurality of sensors may include a layer of acoustically insulating material having a plurality of predetermined mounting locations for the plurality of sensors. The plurality of predetermined mounting locations may define a pattern within an area defined by a perimeter of the layer of acoustically insulating material. In addition, the flexible carrier may include an adhesive layer adjacent to a side of the layer of acoustically insulating material. The adhesive layer may be adapted to adhere the flexible carrier to a surface of a biological entity.

In accordance with still another aspect of the invention, a sensor for use with a biological entity may include a sealed housing adapted to be disposed within the biological entity. The sensor may also include an acoustic transducer disposed within the sealed housing and adapted to receive a sound from the biological entity through the sealed housing and generate an electrical signal therefrom. Additionally, the sensor may include circuitry coupled to the acoustic transducer that is adapted to amplify the electrical signal.

### BRIEF DESCRIPTION OF THE DRAWINGS

FIG. 1 is a cross-sectional view of an example of a sensor that may be used to simultaneously sense biological sounds and electric potentials produced by a biological entity;

FIG. 2a is a cross-sectional view that depicts another manner in which the electrode of the sensor shown in FIG. 1 may be configured;

FIGS. 2b-2f are plan views that depict other electrode configurations that may be used in connection with the sensor shown in FIG. 1;

FIG. 3 is a cross-sectional view that depicts one manner in which the diaphragm of the sensor shown in FIG. 1 may be coupled to an acoustic transducer via a rigid member;

FIG. 4 is a cross-sectional view of an example of a flexible sensor assembly that can be attached to a surface of a biological entity;

FIGS. 5a-5c are plan views that depict additional examples of flexible sensor assemblies; and

FIG. 6 is a plan view of an example of an acoustic sensor that can be ingested or inserted into a patient.

### DESCRIPTION

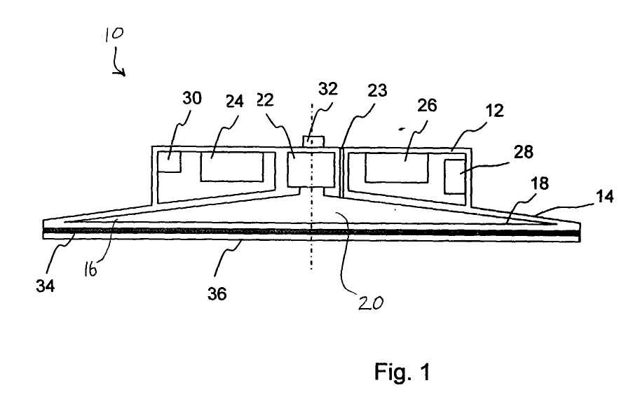

FIG. 1 is a cross-sectional view of an example of a sensor 10 that may be used to simultaneously detect biological sounds and electric potentials produced by a biological entity such as, for example, a human or animal subject. As shown in FIG. 1, the sensor 10 includes a housing 12 having a bell-shaped portion or horn 14 that includes an aperture 16. A diaphragm 18 may cover the aperture 16 to form a chamber 20 to which an acoustic transducer 22 is coupled. A vent passage or aperture 23 may couple the chamber 20 to the ambient pressure surrounding the sensor 10 to eliminate static pressure differentials between the chamber 20 and the ambient surrounding the sensor 10. Circuitry 24 and a power source 26 may be disposed within the housing 12. A connector 28 may also be integrated with the housing 12 to facilitate the connection of the sensor 10 to a processing unit (not shown) such as a personal computer and/or to external sources of power (also not shown).

The sensor 10 may further include a second acoustic transducer 30 for detecting ambient sounds or noise, which may be used to eliminate or reduce noise detected by the acoustic transducer 22. A switch 32 for activating the sensor 10 may also be provided to enable a user such as, for example, a physician, to selectively activate the sensor 10. In addition to the acoustic transducers 22 and 30, the sensor 10 also includes an electrode 34 for measuring electric potentials on a surface of a biological entity. An adhesive layer 36 may be provided to facilitate attachment or coupling of the sensor 10 to a surface of a biological entity.

In general, the sensor 10 may be coupled (e.g., adhered via the adhesive layer 36) to a surface of a biological entity, such as, for example, the skin of a human patient. In this manner, the acoustic transducer 22 may be used to detect sounds or vibrations associated with the region or surface to which the sensor 10 is coupled. Such sounds or vibrations may be generated by organs and/or other biological structures that are underlying or proximate to the region or surface to which the sensor 10 is coupled. In addition, because the electrode 34 is integral with the sensor 10, electric potentials associated with the surface or region of the patient to which the sensor 10 is coupled can be measured by the electrode 34, if desired, while sounds or vibrations associated with that region are detected by the acoustic transducer 22.

The housing 12 may be integrally formed using a molded thermoplastic material or the like, or any other suitable material. Preferably, the material used for the housing 12 is lightweight, durable, electrically insulating and inexpensive. As shown in FIG. 1, the housing 12 may be formed as a substantially unitary or completely unitary (i.e., a one-piece) structure so that the horn or bell 14 is integrally formed with the housing 12. Likewise, some portion of the diaphragm 18 or the entire diaphragm 18 may be integrally molded with the housing 12 or may be a separate structure that is permanently attached to the housing 12 via glue, ultrasonic welding or any other suitable fastening technique. Alternatively, the horn or bell 14 and/or the diaphragm 18 may be removable or detachable to permit disposal and replacement of these parts following each use, if desired. In this manner, the sensor 10 may be reused without requiring cleaning or sterilization of the parts in contact with the patient between uses and the number and cost of components that must be disposed after each use may be minimized. Of course, in the event that the horn or bell 14 and/or the diaphragm 18 are not removable and replaceable, the entire sensor 10 may be cleaned or sterilized or, if desired, discarded after use.

As shown in FIG. 1, the diaphragm 18 completely covers the opening or aperture 16 of the bell portion 14, thereby preventing fluids and other materials or contaminants from entering the aperture 16 and contacting the transducer 22 and/or other sensitive components within the sensor 10. In addition, substantially sealing or covering the aperture 16 with the diaphragm 18 forms the chamber 20, which provides an acoustic load to improve acoustic coupling of the transducer 22 to the diaphragm 18 and the surface to which the sensor 10 is coupled. Improved acoustic coupling may improve the acoustic sensitivity of the sensor 10. However, completely sealing or isolating the chamber 20 from ambient pressure surrounding the sensor 10 may result in static pressure differences that generate a net force or bias on the transducer 22. Such a bias may adversely affect the dynamic range and/or sensitivity of the transducer 22 and, in some cases, may permanently impair the operation of the transducer 22.

To eliminate the development of static pressure differences and the mechanical bias produced thereby, the vent passage 23 extends from the chamber 20 to the ambient surrounding the sensor 10. The cross-sectional area, geometry and length of the passage 23 are preferably configured to optimize the acoustic load or impedance provided by the diaphragm 18 and the chamber 20 over the frequency range of interest (e.g., 1 Hz to 2000 Hz).

The shape and volume of the chamber 20, the material (e.g., fluid or gas) occupying the chamber 20 as well as the thickness, material compliance, shape, etc. of the diaphragm 18 are preferably selected to optimize the acoustic impedance of the sensor 10. More particularly, the acoustic impedance of the sensor 10 is preferably optimized to match the impedance of the surface to which the sensor 10 is coupled. For example, in the case where the sensor 10 is coupled to the skin of a human patient on a particular region of the patient having a particular impedance, the acoustic impedance of the sensor 10 may be matched to that particular impedance to maximize the acoustic power transferred to the transducer 22. More specifically, the surface impedance encountered at an abdominal region of a patient may be different than the surface impedance encountered at a chest region of the same patient because the abdominal region is typically more compliant (i.e., elastic) than the chest region. Thus, different specific configurations (i.e., diaphragm types, chamber geometries and fill materials, bleed passage configurations, etc.) of the sensor 10 may be used to best suit the region of the patient to which the sensor 10 is to be coupled. In the case where a portion of the sensor 10 is detachable and/or disposable such as, for example, the horn or bell 14 and/or diaphragm 18, a variety of differently configured detachable parts may be provided to enable selection and attachment of the particular part or parts that will provide the best acoustic impedance for measuring sounds emanating from a particular area or region of a particular type of patient, which may be a human subject, an animal subject or some other type of biological entity.

While the chamber 20 of the example sensor 10 shown in FIG. 1 is filled with air, the chamber 20 can instead be filled with any other fluid or gas to achieve a desired acoustic impedance or any other acoustic property. In the case where the chamber 20 is filled with a fluid, the passage 23 may be elongated to prevent fluid from exiting the sensor 10 and/or to prevent air from being drawn into the chamber 20 and mixed with the fluid therein. Alternatively, a flexible member (e.g., a membrane or panel) disposed between the chamber 20 and the ambient surrounding the sensor 10 may be used to enable controlled expansion and contraction of fluid within the chamber 20. For example, the diaphragm 18, the bell or horn 14, and/or any other part of the sensor housing 12 forming a boundary between the fluid within the chamber 20 and the ambient surrounding the sensor 10, may be used to provide the compliant member.

The acoustic transducer 22 is preferably an electret microphone. An electret microphone provides a relatively inexpensive transducer that provides a high sensitivity and desirable noise characteristics. However, other types of transducers such as, for example, accelerometers, capacitive microphones, other capacitive transducers, ultrasonic sensors, optical transducers, etc. may be used instead. Sounds or vibrations imparted by, for example, a patient's skin to the diaphragm 18 are propagated through the chamber 20 to the acoustic transducer 22.

The sounds received by the acoustic transducer 22 are converted into electrical signals that may be processed by the circuitry 24 prior to being conveyed to a processing system or the like via the connector 28. In particular, the circuitry 24 may include signal conditioning circuitry such as, for example, amplification circuitry, filtering circuitry which may be used to eliminate noise, prevent aliasing, etc., safety-related circuitry for preventing patients from being shocked and/or for protecting the various electronic components within the sensor 10 from electrostatic discharges, power surges, etc., or any other desired circuitry. The circuitry 24 may be implemented using one or more printed circuit boards, hybrid circuits, integrated circuits, passive and active analog circuit components, digital processors and/or other digital components, etc. Furthermore, while the circuitry 24 is shown in FIG. 1 as being disposed within the housing 12, some or all of the circuitry 24 may instead be external to the sensor 10 and may be coupled to the sensor 10 via wires, wireless communications, a combination of hardwired and wireless communications, or via any other desired technique or combination of techniques.

In addition to receiving and processing electrical signals produced by the acoustic transducer 22, the circuitry 24 may also receive and process electrical signals produced by the acoustic transducer or environmental noise sensor 30. More specifically, the circuitry 24 may use the signals received from the environmental noise sensor 30 to perform adaptive filtering techniques and/or to perform noise subtraction techniques, such as, for example, subtraction of a multiple of the environmental noise signal from the skin surface signals received from the acoustic transducer 22, that improve the quality of the signals received from the acoustic transducer 22.

The power source 26 may be any suitable circuitry for providing power to the circuitry 24 and, if needed, to the transducers 22 and 30. The power source 26 may be a regulated direct current (DC) supply, a battery (rechargeable or disposable), an unregulated power conversion circuit, or any other suitable source of power. As with the circuitry 24, the power source 26 may be external to the sensor 10 and, in that case, power may be conveyed to the circuitry 24 and the transducers 22 and 30 via the connector 28 and/or via separate wires.

The connector 28 may be implemented using a plurality of screw terminals and/or one or more modular connectors. The connector 28 preferably facilitates attachment of the power and other signal wires to the sensor 10. In addition to facilitating connections between the sensor 10 and an external computer system or processor, the connector 28 may also provide a water-proof or splash-proof connection so that in the event fluids are emitted or present during use of the sensor 10, these fluids cannot foul the electrical connections within the connector 28. Alternatively, the connector 28 may be remotely situated from the sensor 10 and coupled to the sensor 10 via one or more wires.

The adhesive layer 36 may cover all or some of the area defined by the diaphragm 18. The adhesive layer 36 is preferably made of an adhesive that provides suitable electrical conductivity and acoustic transmission properties, thereby minimizing the effect that the adhesive layer 36 may have on the overall sensitivity of the sensor 10. In addition, the adhesive layer 36 is preferably made of an adhesive material that facilitates easy attachment or coupling of the sensor 10 to the skin of a patient. Of course, different materials may be used for the adhesive layer 36 to optimize attachment or coupling of the sensor 10 to different types of biological entities. For example, certain adhesive materials may provide optimal attachment properties when used with human patients on human skin, while other adhesive materials may provide optimal attachment properties when used with animal subjects and the different types of surfaces encountered with such subjects (e.g., fur, scales, etc.).

The electrode 34 is formed using a conductive material that covers a portion or all of the surface area defined by the diaphragm 18 and is configured to directly contact the surface of a biological entity such as, for example, the skin of a human patient. The electrode 34 may be coupled to the circuitry 24 and the connector 28 via wires or other conductive elements, none of which are shown for purposes of clarity. Thus, electrical potentials generated on the skin of a patient adjacent to the electrode 34 are conveyed to the circuitry 24 for signal conditioning and in turn may be passed via the connector 28 to a display (not shown) and/or a processing unit (e.g., a computer) for analysis. In particular, signals associated with detected electrical potentials may be used to perform electrocardiograms, electrogastrograms, electroencephalograms, or any other desired analyses or diagnostic techniques.

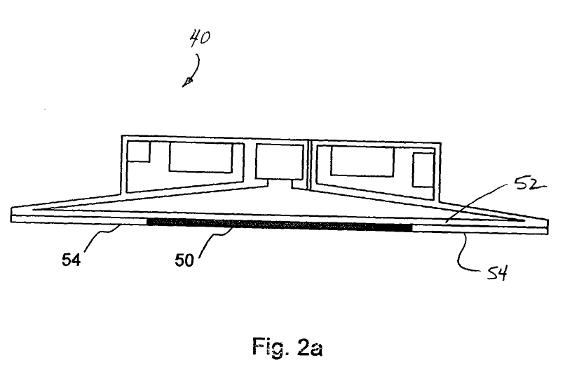

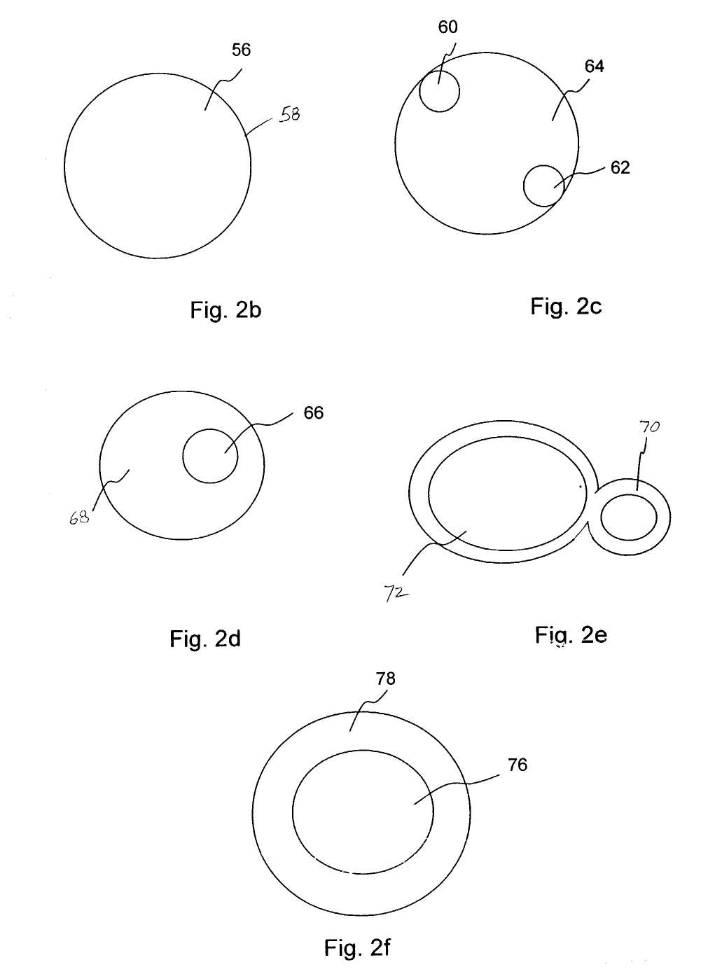

FIGS. 2a-2f depict possible example electrode configurations or geometries. In particular, FIG. 2a depicts a sensor 40, which is similar to the sensor 10 shown in FIG. 1 except that the sensor 40 has an electrode 50 that is approximately centrally disposed on a diaphragm 52 and which is surrounded by an adhesive layer 54. FIG. 2b depicts an electrode 56 that completely covers the area of a diaphragm 58, as is the case in the example shown in FIG. 1. FIG. 2c depicts an electrode composed of two areas 60 and 62 that are adjacent to the outer edge of a diaphragm 64. FIG. 2d depicts an electrode 66 that is spaced from the centroid of the area defined by a diaphragm 68. FIG. 2e depicts an electrode 70 that does not overlap a diaphragm 72. FIG. 2f depicts an electrode 76 that is concentrically disposed on a diaphragm 78. As can be seen from FIGS. 2a-2f, a variety of electrode configurations may be used to implement the principals described herein.

While the diaphragm 18 shown in FIG. 1 is depicted as approximately circular and, thus, symmetrical about an axis, other non-circular geometries and/or geometries that are non-symmetrical about an axis could be used instead. For example the diaphragm 72 shown in FIG. 2e depicts one example of such a non-circular and non-symmetrical diaphragm configuration. More generally, the geometry of the diaphragm 18 may be approximately oval in shape, polygonal, or any other desired shape. Likewise, while the horn or bell 14 is depicted in FIG. 1 as having a conical shape, any other shape such as, for example, a cylindrical or pyramidal shape could be used instead.

A particular combination of electrode, diaphragm and housing configurations may be employed to optimally detect sounds and/or electric potentials associated with a particular region of a biological entity. For example, one set of shapes or geometries may be ideal for detecting sounds emanating from a patient's gastrointestinal system and another set of shapes or geometries may be better suited for detecting sounds emanating from a patient's pulmonary system.

Although the acoustic transducer 22 and the diaphragm 18 are depicted in FIG. 1 as separate components, these components may be integrated or combined within a single structure or member. For example, the diaphragm 18 may be made from a piezoelectric material such as a metallized piezoelectric thin film that generates electrical signals in response to pressures produced by sounds or vibrations. In the case where a diaphragm is made of a piezoelectric material, the manner in which the diaphragm is fixed to a housing and the thickness of the diaphragm affects the acoustic sensitivity of the diaphragm. For example, a relatively high sensitivity can be achieved by using a thin diaphragm that is mounted or otherwise fixed to the housing only at its edges so that the diaphragm is subjected to bending stresses. An air cavity, such as the chamber 20 shown in FIG. 1, located opposite a surface of a piezoelectric diaphragm that is in contact with a patient's skin permits the diaphragm to deflect into the air cavity or chamber in response to sounds or vibrations, thereby subjecting the diaphragm to bending stresses.

A piezoelectric diaphragm may also be mounted so that the entire area of the diaphragm that is not in contact with a patient's skin is supported (i.e., rigidly backed or fixed in place), which results in the diaphragm being subjected to primarily axial (e.g., compression or tension) stresses. Still further, partial compliant support of a piezoelectric diaphragm may be used to cause the diaphragm to respond to vibrations or sounds with a combination of bending and axial stresses.

While the above-described examples of the combined acoustic and electric potential sensor include a diaphragm, the diaphragm may be eliminated. In the case where the diaphragm is eliminated, the electrode 34 may, for example, be placed directly on the rim of the bell or horn 14 so that the transducer 22 is directly responsive to sounds traveling from the patient's skin up through the inside of the bell or horn 14. Of course, elimination of the diaphragm 18 may expose the acoustic transducers 22 and 30, the circuitry 24 and the power source 26 to fluids and other contaminants associated with the biological entity to which the sensor 10 is coupled.

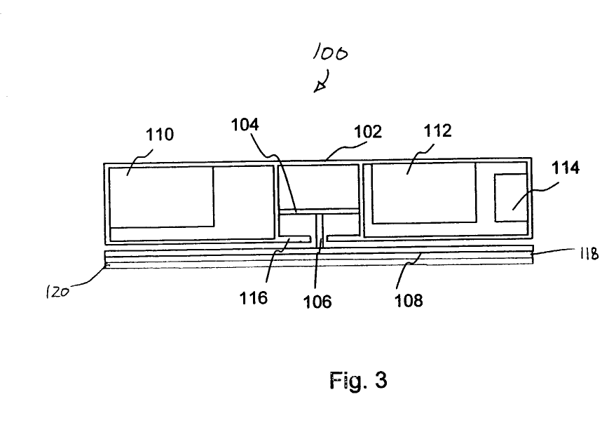

FIG. 3 is a cross-sectional view that depicts another sensor 100 that may be coupled to the surface of a biological entity to detect sounds and/or electric potentials associated therewith. As shown in FIG. 3, the sensor 100 includes a housing 102, an acoustic transducer 104, a rigid member or pushrod 106 that mechanically couples the acoustic transducer 104 to a diaphragm 108, circuitry 110, a power source 112 and a connector 114. A baffle or cover 116 may be included as shown to prevent fluids and other liquid or solid contaminants from fouling the acoustic transducer 104, circuitry 110, power source 112, etc. An electrode 118 and an adhesive layer 120 may also be included as shown in FIG. 3.

Generally speaking, the sensor 104 shown in FIG. 3 is similar to the sensor 10 shown in FIG. 1. However, the sensor 104 shown in FIG. 3 employs a rigid coupling between its diaphragm 108 and acoustic transducer 104, whereas the sensor 10 shown in FIG. 1 employs a fluid or viscous coupling between its acoustic transducer 22 and diaphragm 18. In any event, the acoustic transducer 104 shown in connection with the sensor 100 of FIG. 3 may be made of a piezoelectric material or any other material that generates an electrical signal in response to vibrations imparted via the diaphragm 108 and the rigid member or pushrod 106. To optimize the sensitivity of the sensor 100, the geometries and masses of the oscillating members (e.g., the diaphragm 108, the rigid member 106 and the acoustic transducer 104) are selected to provide an acoustic impedance that most closely matches the impedance of the surface to which the sensor 100 is coupled.

In operation, the sensors 10 and 100 described above may, for example, be attached (i.e., adhered via the respective adhesive layers 36 and 120) to the skin of an animal or human subject to simultaneously detect sounds and electric potentials associated therewith. The detected electric potentials may be used to perform diagnostic procedures such as, for example, electrocardiograms, electrogastrograms, electroencephalograms, etc., whereas the detected sounds may be used to perform various diagnostic techniques using sounds emanating from specific organs, the gastrointestinal system, pulmonary or cardiovascular systems, joints, etc.

As biological sounds travel from their point of origin within a body to a detection point, which is usually at a surface of the body, the sounds typically encounter a wide range of tissue types, each of which may have different acoustic properties. As a result, the sounds associated with a given biological function may appear to have different properties depending on the location of the detection point. Thus, a plurality of detection points (i.e., affixation of a plurality of sensors to a body) may be used to provide improved signal characterization and sound localization. In other words, assessment or diagnosis of a particular biological sound may be facilitated or improved by using information gathered substantially simultaneously from a plurality of acoustic sensors distributed over a region of a body associated with that biological sound. However, the number of sensors used and the specific distribution of the sensors may vary with the intended application. For example, in the case of a pathology that occurs within a relatively small or localized area such as a carotid bifurcation, a few or possibly a single sensor may be sufficient. On the other hand, detection of an abdominal or pulmonary pathology may require a larger number of sensors distributed over a larger region. For example, it may be beneficial to distribute a relatively large number of sensors over a chest region of a human patient to enable an effective diagnosis of a pneumothorax condition.

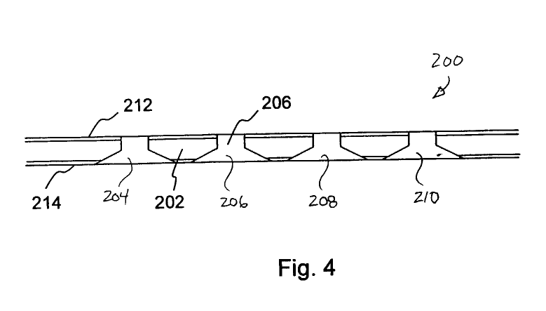

FIG. 4 is a cross-sectional view that depicts one manner in which a plurality of sensors (such as, for example, sensors for detecting biological sounds and/or electrical potentials similar or identical to those described herein) may be arranged in an array to facilitate placement of a plurality of sensors over a region of a patient's body. As shown in FIG. 4, a flexible sensor array 200 includes a flexible carrier or pad 202 that holds a plurality of sensors 204-210 in a predetermined pattern or array geometry. A vinyl backing 212 may be affixed to the carrier or pad 202 to provide increased tear and puncture resistance and an adhesive layer 214 may be included to facilitate attachment of the sensor array 200 to, for example, the skin of a human patient. Inclusion of the adhesive layer 214 may eliminate the need to include an adhesive layer (e.g., similar or identical to the adhesive layers 36, 54 and 120 shown in FIGS. 1, 2a and 3) on the sensors 204-210.

The flexible carrier or pad 202 is made of a lightweight material having good acoustic insulation properties that prohibit the efficient transmission of sound waves or vibrations. As a result of the acoustic insulation properties of the pad 202, each of the sensors 204-210 senses vibrations or sounds that directly underlie it and does not receive vibrations or sounds associated with the area covered by another sensor. In other words, the pad 202 may be used to substantially minimize cross-talk between the sensors 204-210. The flexible carrier or pad 202 may, for example, be made of an open-cell polyurethane foam sheeting and may have a thickness of about one-quarter of an inch and a density of about two pounds per cubic foot. However, other types of materials such as, for example, closed-cell foams having different thicknesses and densities may be used instead.

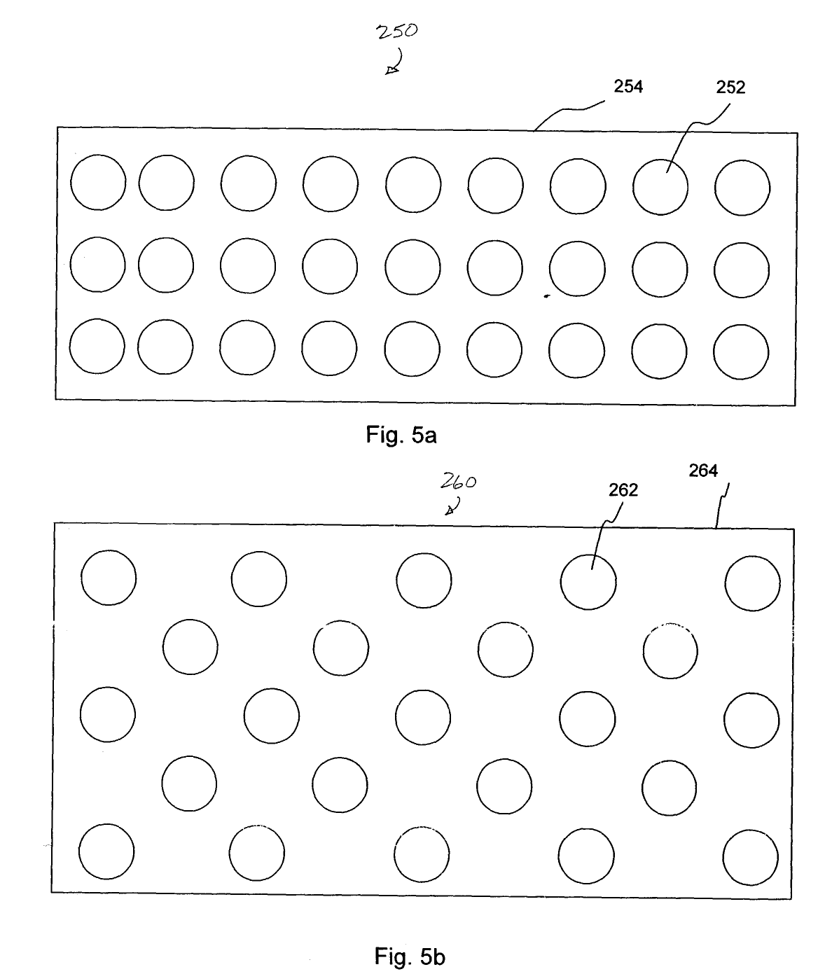

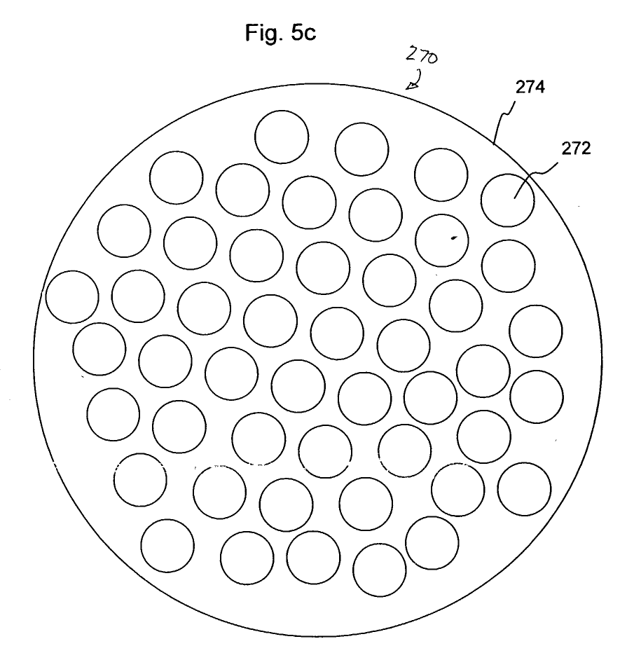

FIG. 5a is a plan view of an example of a flexible sensor array 250 that may have a cross section similar or identical to that of the array 200 shown in FIG. 4. As shown in FIG. 5a, the array 250 includes a plurality of sensors, one of which is indicated at reference numeral 252, that are mounted to a flexible pad or carrier 254 in a grid-like pattern.

FIG. 5b is a plan view of another example of a flexible sensor array 260. As shown in FIG. 5b, the sensor array 260 includes a plurality of sensors, one of which is associated with reference numeral 262, that are mounted in a diagonal grid pattern to a flexible carrier or pad 264.

FIG. 5c is a plan view of yet another example of a flexible sensor array 270. As shown in FIG. 5c, the sensor array 270 includes a plurality of sensors, one of which is associated with reference numeral 272, that are mounted in a honeycomb-like pattern within a circular flexible carrier or pad 274.

The sensors within the arrays shown in FIGS. 4 and 5a-5c may be configured to permit individual or selective activation (i.e., turned on or off) using a variety of techniques. For example, each of the sensors may include a switch such as the switch 32 shown in FIG. 1. The switches may be touch-sensitive so that a user such as, for example, a physician, may activate or turn on the sensor via finger contact. Touch-sensitive switches may be implemented using any desired sensing mechanism including capacitive mechanisms, optical mechanisms, pressure-sensitive mechanisms, and thermal mechanisms. If desired, the touch-sensitive mechanism for each of the touch-activated sensors may be attached to or integrated within the acoustic sensor or, alternatively, may be located adjacent to the sensor within the flexible pad or carrier.

Still other sensor activation techniques may be used with the sensor arrays described herein. For example, an operator may activate sensors falling within a particular region within an array by touching around a perimeter of the region of interest. In this case, a computer system or other processor to which the sensor array is coupled can activate all sensors falling with the touched perimeter and deactivate all other sensors external to that area defined by the perimeter. Additionally or alternatively, sensors within an array could be activated via a remotely situated control system and/or panel. In any event, an indicator such as, for example, a light-emitting diode may be used to indicate the status of each sensor within the array (i.e., whether a sensor is active or inactive).

Still further, in some cases the flexible carrier or pad may not be pre-populated with sensors and a desired number and type of sensors may be installed in a desired pattern in a desired location within the pad to suit a particular application. Alternatively, the pad may be populated with all of the disposable sensor components and the reusable parts may be added as needed. In any case, the flexible carrier or pad may be used as a template or guide to facilitate sensor placement.

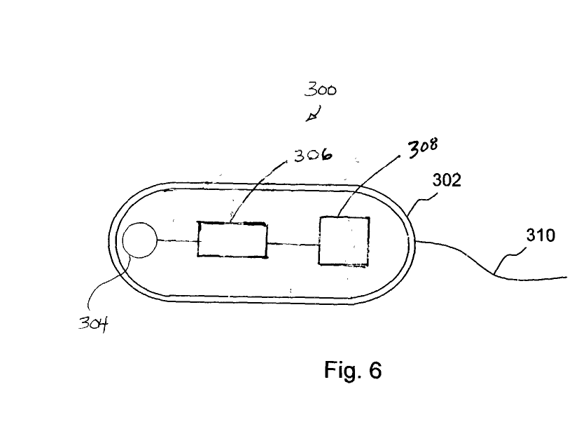

FIG. 6 is a plan view of an example of an acoustic sensor 300 that may be ingested or otherwise inserted into a human or animal body. As shown in FIG. 6, the sensor 300 may include a capsule-shaped housing 302, an acoustic sensing element or transducer 304, circuitry 306, a power source 308 and electrical wires or leads 310.

The capsule-shaped housing 302 may be made of a molded plastic material that can tolerate the conditions typically present within a human or animal body. In addition, the housing 302 is made of a material that provides good acoustic transmission properties and, thus, can readily transmit sounds external to the housing 302 through the housing 302 to the sensing element or acoustic transducer 304. The sensing element 304 may be any desired type of acoustic transducer such as, for example, an electret microphone.

The circuitry 306 may include signal conditioning circuitry such as, for example, amplification circuitry, filtering circuitry, protection circuitry, etc. The circuitry 306 may be implemented using any desired combination of digital or analog circuitry using any combination of discrete components, hybrid circuits, integrated circuits, etc.

The power source 308 may be a battery, a piezoelectric power source that coverts vibrations of the sensor 300 into electrical power, a thermocouple-based device, a mechanical device driven by a fluid flow external to the sensor 300, or any other desired power source. The power source 308 provides power to the circuitry 306 and directly or indirectly to the acoustic sensing element or acoustic transducer 304. While the power source 308 is shown as being disposed within the sensor 300, the power source 308 could instead be external to the sensor 300.

One or more sensors such as the sensor 300 shown in FIG. 6 may be inserted, ingested, or otherwise located within a human or animal body to detect sounds therein. For example, a human patient may swallow the sensor 300 and the acoustic information detected by the sensor 300 may be used to diagnose a variety of gastrointestinal pathologies such as, for example, delayed gastric emptying. The wires 310 may function as a tether that enables repositioning and/or removal of the sensor 300 after insertion, ingestion, etc.

The example sensor 300 shown in FIG. 6 is depicted as using the wires 310 to convey signals containing acquired acoustic information from the sensor 300 to a monitoring device such as, for example, a personal computer or any other processor, neither of which are shown, via the wires 310. However, the acoustic information could instead be transmitted using a wireless transmission technique, in which case the wires 310 may be eliminated (in the case where the power source 308 is internally disposed as shown in FIG. 6). When configured for wireless transmission, a string, a small diameter wire or the like may be used to function as a positioning and/or removal tether, if desired.

While the invention has been described with reference to specific examples, which are intended to be illustrative only and not to be limiting of the invention, it will be apparent to those of ordinary skill in the art that changes, additions or deletions may be made to the disclosed embodiments without departing from the spirit and the scope of the invention.

What is claimed is:

1. A sensor for use with a biological entity, comprising:
a housing;
a first acoustic transducer disposed within the housing and adapted to detect a biological sound impinging on a surface of the biological entity; and
an electrode integral with the sensor and adapted to detect an electric potential associated with the surface of the biological entity.

2. The sensor of claim 1, further including a diaphragm adapted to convey the biological sound to the first acoustic transducer.

3. The sensor of claim 2, further including a rigid member that couples the diaphragm to the first acoustic transducer.

4. The sensor of claim 2, wherein the diaphragm is fixed to the housing and extends over an aperture of a bell-shaped portion of the housing

5. The sensor of claim 2, wherein the electrode is disposed on a surface of the diaphragm.

6. The sensor of claim 5, wherein the electrode covers less than the entire surface of the diaphragm.

7. The sensor of claim 2, wherein the first acoustic transducer is integral with the diaphragm.

8. The sensor of claim 7, wherein the diaphragm is made of a piezoelectric material.

9. The sensor of claim 2, further including a chamber that is disposed between the diaphragm and the first acoustic transducer.

10. The sensor of claim 1, further including an adhesive portion adapted to facilitate attachment of the sensor to the surface of the biological entity.

11. The sensor of claim 10, wherein the adhesive portion is adapted to transmit acoustic energy from the surface of the biological entity to the first acoustic transducer.

12. The sensor of claim 1, further including a second acoustic transducer that is adapted to detect a sound associated with the ambient surrounding the biological entity.

13. The sensor of claim 12, further including circuitry that subtracts a first signal associated with the sound associated with the ambient surrounding the biological entity from a second signal associated with an output of the first acoustic transducer.

14. The sensor of claim 1, further including a switch adjacent to the housing that enables selective activation of the sensor.

15. The sensor of claim 14, wherein the switch is touch-sensitive.

16. The sensor of claim 1, wherein the sensor is adapted to provide a first acoustic impedance substantially equal to a second acoustic impedance associated with the surface of the biological entity.

17. The sensor of claim 1, wherein at least a portion of the housing is adapted to be removed.

18. The sensor of claim 1, wherein the housing includes a bell-shaped portion extending between the first acoustic transducer and the electrode.

19. A sensor assembly for use with a biological entity, comprising:
a flexible carrier that is adapted to be attached to a surface of the biological entity; and
a plurality of acoustic sensors fixed to the flexible carrier and defining a pattern within an area defined by a perimeter of the flexible carrier, wherein each of the acoustic sensors is adapted to detect a sound at the surface of the biological entity.

20. The sensor assembly of claim 19, wherein the flexible carrier is made of a foam material having an acoustic insulation property.

21. The sensor assembly of claim 19, wherein the flexible carrier includes a backing layer.

22. The sensor assembly of claim 19, wherein the flexible carrier includes an adhesive layer.

23. The sensor assembly of claim 19, wherein the pattern is one of a grid-like pattern and a honeycomb pattern.

24. The sensor assembly of claim 19, further including a plurality of touch-sensitive areas, each of which is uniquely associated with one of the acoustic sensors, wherein each of the touch-sensitive areas is adapted to control activation of its respective one of the acoustic sensors.

25. The sensor assembly of claim 24, wherein a group of the touch-sensitive areas are adapted to control the activation of ones of the acoustic sensors that lie within a perimeter defined by the group of the touch-sensitive areas.

26. The sensor assembly of claim 19, wherein each of the acoustic sensors further includes an electrode adapted to detect an electric potential at the surface of the biological entity.

27. The sensor assembly of claim 19, further including a plurality of light emissive devices, each of which is uniquely associated with one of the plurality of acoustic sensors and which is adapted to emit light to indicate that its respective acoustic sensor is active.

28. A flexible carrier for holding a plurality of sensors, the flexible carrier comprising:
a layer of acoustically insulating material having a plurality of predetermined mounting locations for the plurality of sensors, wherein the plurality of predetermined mounting locations define a pattern within an area defined by a perimeter of the layer of acoustically insulating material; and
an adhesive layer adjacent to a first side of the layer of acoustically insulating material, wherein the adhesive layer is adapted to adhere the flexible carrier to a surface of a biological entity.

29. The flexible carrier of claim 28, further including a backing layer adjacent to a second side of the layer of acoustically insulating material.

30. The flexible carrier of claim 28, wherein the layer of acoustically insulating material is a polyurethane foam material.

31. The flexible carrier of claim 28, wherein each of the plurality of predetermined mounting locations includes an opening in the layer of acoustically insulating material.

32. The flexible carrier of claim 31, wherein each of the plurality of predetermined mounting locations further includes a disposable sensor component adapted to cooperatively engage with one of the plurality of sensors.

33. The flexible carrier of claim 28, wherein the pattern defined by the plurality of predetermined mounting locations is one of a grid pattern and a honeycomb pattern.

34. The flexible carrier of claim 29, wherein the backing layer is made of a vinyl material.

35. A sensor for use with a biological entity, comprising:
a sealed housing adapted to be disposed within the biological entity;
an acoustic transducer disposed within the sealed housing and adapted to receive a sound from the biological entity through the sealed housing and to generate an electrical signal therefrom;
circuitry coupled to the acoustic transducer, wherein the circuitry is adapted to amplify the electrical signal; and
a tether coupled to the housing.

36. The sensor of claim 35, wherein the sealed housing is adapted to be ingested by the biological entity.

37. The sensor of claim 35, wherein the sealed housing has a capsule-shaped profile.

38. The sensor of claim 35, wherein the acoustic transducer is a microphone.

39. The sensor of claim 35, wherein the tether includes a wire for conveying an electrical signal.

40. The sensor of claim 35, further including a power source disposed within the sealed housing.
